(19) United States
(12) Patent Application Publication
(10) Pub. No.: US 2020/0066383 A1
(43) Pub. Date: Feb. 27, 2020

(54) INTERACTIVE HEALTH CARE PLANS AND RELATED METHODS AND SYSTEMS

(71) Applicant: HealthTap, Inc., Mountain View, CA (US)

(72) Inventors: Geoffrey W. Rutledge, Palo Alto, CA (US); Ron J. Gutman, Atherton, CA (US); Sean K. Mehra, San Francisco, CA (US)

(21) Appl. No.: 16/549,899

(22) Filed: Aug. 23, 2019

Related U.S. Application Data
(60) Provisional application No. 62/722,044, filed on Aug. 23, 2018.

(51) Int. Cl. G16H 20/00; G16H 10/60; G16H 70/20; G16H 50/70; G16H 50/30; G09B 5/02; H04L 29/08

(52) U.S. Cl. CPC G16H 20/00; G16H 10/60; G16H 70/20; H04L 67/12; G16H 50/30; G09B 5/02; G16H 50/70

(57) ABSTRACT

A system for providing an interactive care plan to a patient is described. A patient computer device receives symptom information for the patient. A server hosts a virtual consultation application. The virtual consultation application receives from the patient computer device symptom information for the patient; accesses interactive care plans each specifying one or more measures to be taken on a patient's behalf; uses the received symptom information for the patient to select one of the plurality of accessed interactive care plans; and recommends the selected interactive care plan to the patient via the patient computer device.

### CROSS-REFERENCE TO RELATED APPLICATIONS

This Application claims the benefit of provisional U.S. Application No. 62/722,044, filed Aug. 23, 2018 and entitled “Interactive Health Care Plans and Related Methods and Systems,” which is hereby incorporated by reference in its entirety.

This application is also related to U.S. patent application Ser. No. 15/298,099, filed Oct. 19, 2016, and which is a Continuation-in-Part of U.S. patent application Ser. No. 14/320,241, filed Jun. 30, 2014, which claims priority to U.S. Provisional Application No. 61/841,145, filed Jun. 28, 2013, U.S. Provisional Application No. 61/841,151, filed Jun. 28, 2013 and U.S. Provisional Application No. 61/841,174, filed Jun. 28, 2013, the contents of each of which is incorporated herein by reference.

In cases where the present application conflicts with a document incorporated by reference, the present application controls.

### BACKGROUND

When patients seek medical care for health issues, they typically visit a doctor's office or urgent care center, undergo baseline testing, describe the symptoms of their health issues to a medical professional, and receive a care plan for treatment of their issues. The care plan is generally based on industry-accepted standard methods, often referred to as “pathways,” “protocols,” “care process models,” or “standards of care.” Care plans may be determined based on symptoms presented, the patient's medical history, risk factors, and other considerations.

### BRIEF DESCRIPTION OF THE SEVERAL VIEWS OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a box diagram showing the computer-implemented system for providing interactive care plans to a patient from a health care provider located remote from the patient, in accordance with a first exemplary embodiment of the present disclosure.

FIG. 2A is a flow chart for an exemplary interactive care plan based on symptom severity, according to the first embodiment of the present disclosure.

FIG. 2B is a flow chart for an exemplary interactive care plan for a pregnant patient who is pre-diabetic.

FIG. 2C is a flow chart for an exemplary interactive care plan for a patient with mild Chronic Obstructive Pulmonary Disease.

FIG. 3 is a box diagram showing a computer-implemented system for delivering an interactive care plan to a patient from a health care provider located remote from the patient, according to a second exemplary embodiment of the present disclosure.

FIG. 4 is a flowchart illustrating a method of delivering an interactive care plan to a patient from a health care provider located remote from the patient.

### DETAILED DESCRIPTION

Typical care plans are often maintained on primitive systems—like paper or PDFs—that the inventors have recognized require expensive, inefficient, and manual management by providers and their staffs. The inventors have further recognized that care plans maintained in these media are difficult to share with patients, caregivers, and other medical professionals. They are difficult to amend, and cannot be changed in real-time based on patient performance data. The inventors have determined that this is a meaningful obstacle to providing the timely, individualized care that enhances outcomes and satisfaction. Further, the inventors have recognized that these media do not offer a platform for health care providers to track, analyze, and share patient results and outcomes. This makes it difficult to build consensus for new care plans or develop best practices for individuals and groups. As a result, one-size-fits-all care plans may not reflect the best, newest data available.

In response to recognizing an unaddressed need in the industry to address the aforementioned deficiencies and inadequacies, the inventors have conceived and reduced to practice systems and methods for providing interactive health care plans, described further herein.

Embodiments of the present disclosure provide a system and method for providing interactive care plans to a patient from a health care provider located remote from the patient. Briefly described, in architecture, one embodiment of the system, among others, can be implemented as follows. The system includes at least one patient computer device having a processor and computer-readable memory and at least one interactive care plan. The interactive care plan is located on a remote database. The system also includes a virtual consultation application hosted at least partially on a server, which has a processor and computer-readable memory. The virtual consultation application is electronically accessible over at least one network system by the at least one patient computer device and the remote database. The virtual consultation application receives patient symptom information using the at least one patient computer device. The virtual consultation application also recommends an interactive care plan to the patient that includes at least one of patient education, a virtual consultation with the health care provider, and an in-person consultation with the health care provider.

The present disclosure can also be viewed as providing methods of delivering an interactive care plan to a patient from a health care provider located remote from the patient. In this regard, one embodiment of such a method, among others, can be broadly summarized by the following steps: receiving, in a virtual authoring application, an interactive care plan from the health care provider using a provider computer device, wherein the provider computer device has a processor and computer-readable memory, and wherein the virtual authoring application is hosted at least partially on a first server having a processor and computer-readable memory and electronically accessible over at least one network system; storing the interactive care plan on a remote database electronically accessible over the at least one network system; receiving, in a virtual consultation application, patient symptom information using at least one patient computer device, wherein the at least one patient computer device has a processor and computer-readable memory, and wherein the virtual consultation application is hosted at least partially on a second server having a processor and computer-readable memory and electronically accessible over the at least one network system; matching patient symptom information to a corresponding interactive care plan; and recommending an interactive care plan to the patient, wherein the interactive care plan is at least one of patient education, virtual consultation with a health care provider, and in-person consultation.

Embodiments of the present disclosure provide a system and method for delivering interactive care plans to a patient from a health care provider located remote from the patient. Briefly described, in architecture, one embodiment of the system, among others, can be implemented as follows. The system includes a provider computer device having a processor and computer-readable memory and a remote database. A virtual authoring application hosted at least partially on a server, which has a processor and computer-readable memory, is electronically accessible over at least one network system by the provider computer device and the remote database. The virtual authoring application receives an interactive care plan from the health care provider using the provider computer device, and the interactive care plan is stored on the remote database. The interactive care plan is accessible to a patient from the remote database.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

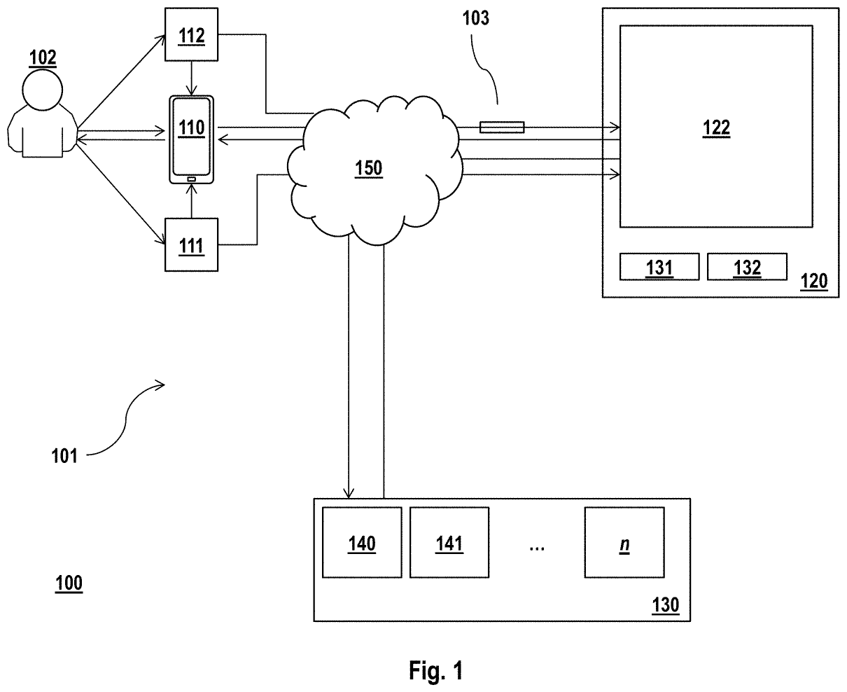

FIG. 1 is a box diagram 100 showing a computer-implemented system for providing interactive care plans to a patient from a health care provider located remote from the patient, in accordance with a first exemplary embodiment of the present disclosure. The system 101 includes at least one patient computer device 110 and at least one interactive care plan 140. The interactive care plan 140 is located on a remote database 130. The system 101 also includes a virtual consultation application 122 hosted at least partially on a server 120, which has a processor 131 and computer-readable memory 132. The virtual consultation application 122 is electronically accessible over at least one network system 150 by the at least one patient computer device 110 and the remote database 130. The virtual consultation application 122 receives patient symptom information 103 using the at least one patient computer device 110. The virtual consultation application 122 also recommends an interactive care plan 140 to the patient 102 that includes at least one of patient education, a virtual consultation with the health care provider, and an in-person consultation with the health care provider (not shown).

The patient computer device 110 may be any electronic computer device that provides an interface between the patient 102 and the virtual consultation application 122. For example, the patient computer device 110 may be a personal computer or a mobile device, such as a tablet or a smartphone. The patient 102 may use these devices to seek a medical consultation from home, work, in public, or in a remote location. In another example, the patient computer device 110 may be a computer kiosk located in a medical facility that would allow the patient to seek a self-service medical consultation. In another example, the patient computer device 110 may be a computer kiosk operated by a medical professional, such as a nurse or technician. The patient computer device 110 may include components generally found in a computer or mobile device, such as a processor, computer-readable memory, screen or monitor, input interface, and network connection hardware. The patient computer device 110 may allow a patient 102, caregiver, or medical professional to interface with the virtual consultation application 122 using a mouse and keyboard, touch interface, audio interface, visual interface, or any combination thereof The patient 102 may use the patient computer device 110 to enter identifying information, such as their name, address, date of birth, and the like. The patient 102 may also use the patient computer device 110 to enter basic health statistics such as their age, weight, height, medical history, and the like.

Additional patient computer devices 111, 112 are shown. These additional devices may be testing or diagnostic equipment capable of performing medical tests on or taking measurements from the patient 102. For example, such equipment may include common medical devices such as cameras, scales, thermometers, blood glucose meters, blood pressure cuffs, heart rate sensors, and the like, which have been connected to the network 150 and may be part of the Internet of Things. The additional patient computer devices 111, 112 may conduct tests or measurements on the patient 102, record any results, and report them to the virtual consultation application 122 via the network 150. In one example, the devices 111, 112 may connect directly to the network 150. In another example, the devices 111, 112 may connect to the patient computer device 110, for example, through Bluetooth®, NFC, Wi-Fi®, or other short-range wireless connection protocols. The devices 111, 112 may conduct tests or take measurements of the patient 102 and send the results to the patient computer device 110. The patient computer device 110 may receive, process, or enter the data through the virtual consultation application 122. For instance, device 111 may be a heart rate monitor connected to patient computer device 110 via Bluetooth®. The heart rate monitor may measure the patient 102's heart rate and transmit the heart rate information to the patient computer device 110. The patient computer device 110 may process the heart rate information and transmit it to the database 130 via the virtual consultation application 122. As another example, device 112 may be a blood glucose monitor with a low-power NFC transmitter embedded in it. A patient 102 may take a blood glucose reading with device 112, scan device 112 using an NFC-enabled phone, and instruct the virtual consultation application 122 to enter the blood glucose reading. Data sent by the devices 111, 112 may be entered into a patient 102's user profile on the virtual consultation application 122. This may occur when the patient 102 is seeking a consultation, and the virtual consultation application 122 prompts the patient to provide measurements or test data. This may also occur when the patient 102 is not seeking a consultation, for example, when a patient 102 conducts blood glucose tests multiple times a day, or when a patient 102 weighs themselves once a week. The devices 111, 112 may operate automatically, or they may require some guidance from the patient 102, a caregiver, or a medical professional. In another example, the additional patient computer devices 111, 112 may be personal computers or mobile devices.

Multiple patient computer devices 110, 111, 112 may be used during a consultation. For example, a patient 102 seeking a consultation in their home may use a personal computer to load the virtual consultation application 122 at home, use a connected thermometer or scale to submit information about themselves, then may seek a video consultation using a smartphone.

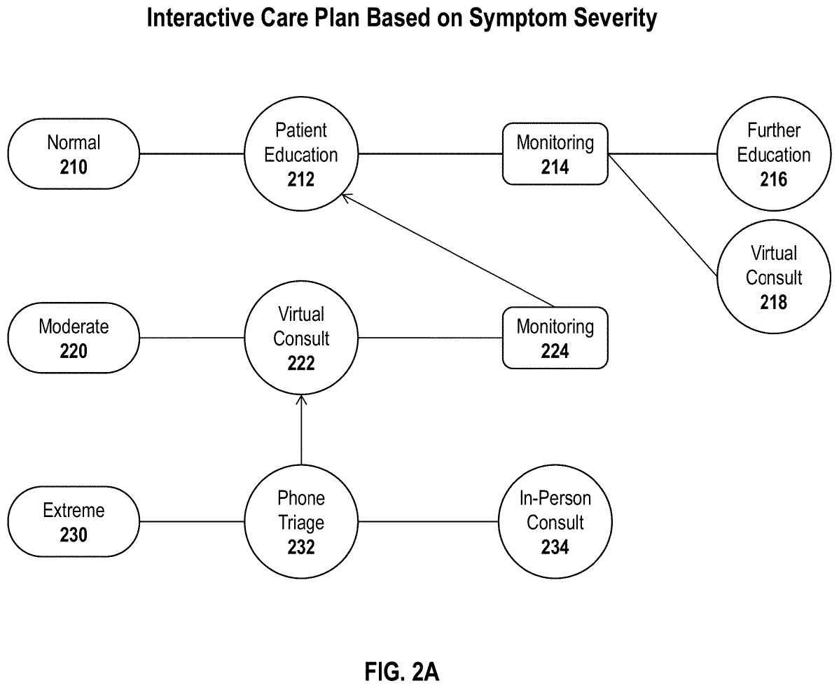

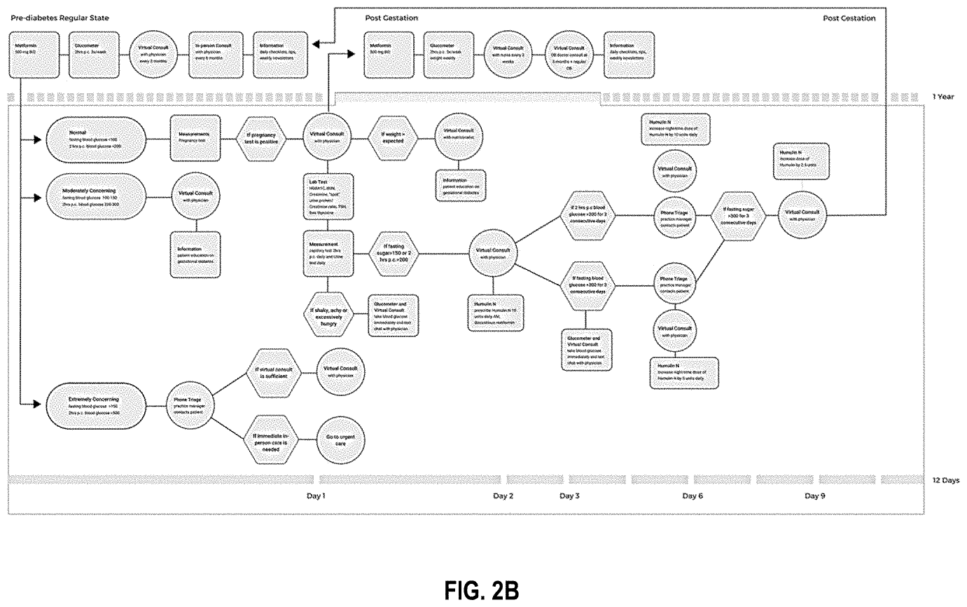

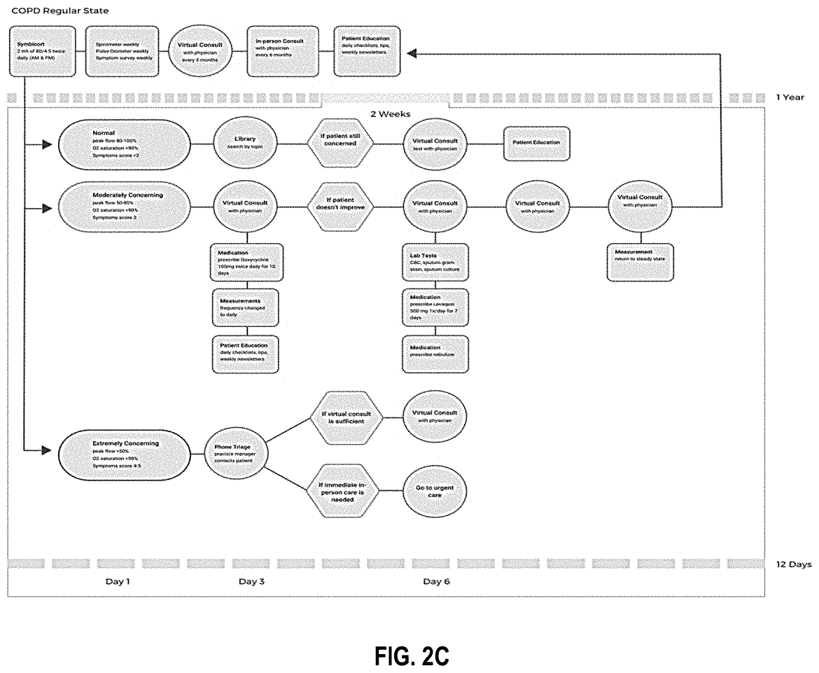

The interactive care plan 140 is a health treatment plan developed by a medical professional. The interactive care plan 140 may set forth one or more treatment options for a patient 102 that may vary depending on a number of factors. For instance, the interactive care plan 140 may provide multiple treatment options depending on the type and severity of symptoms the patient 102 presents with. In another example, the interactive care plan 140 may provide a single treatment option, but may include multiple reminders, notifications, or appointments. This may encourage the patient 102 to follow through with the treatment. Other interactive care plans 140 may be provided based on new or experimental procedures, cost of treatment, or other factors. Interactive care plans are discussed in greater detail in FIG. 2A.

The remote database 130 may store one or more interactive care plans 140. FIG. 1 shows, as an example, multiple interactive care plans 140, 141, and up to any n number of interactive care plans. In one example, the remote database 130 may store hundreds of care plans or more. The remote database 130 may also store additional information, such as patient histories, electronic medical records (EMRs), application preferences, and the like. The remote database 130 may include components generally found on database storage equipment, such as a processor and computer-readable memory. The remote database 130 may also include a power source and a network connection. The remote database may be a cloud database, as it may be hosted in any location and accessible to users over the network system 150.

The system 101 also includes a server 120 having a processor 131 and computer-readable memory 132. The server 120 may be any computer server that is typically used to host software applications and may include a power source, and a network connection.

The virtual consultation application 122 (hereinafter “consultation application”) may be a software application hosted at least partially on the server 120. The consultation application 122 may provide a user interface, including a graphical user interface, for the patient 102 when seeking a consultation.

The consultation application 122 may be accessible over at least one network system 150 by the at least one patient computer device 110 and the remote database 130. The network system 150 may be any network available to the patient 102. In one example, the network system 150 may be the Internet. In another example, the network system 150 may be a closed wired or wireless network, such as an intranet, LAN, WLAN, or cellular network. In one example, the consultation application 122 may be accessible by multiple network systems 150, both public and private. The consultation application 122 may utilize encryption protocols over the network system 150, such as Transport Layer Security (TLS) or Secure Sockets Layer (SSL), to establish a secure connection and limit access to sensitive medical information. The network system 150 may additionally be connected to and accessible by medical facilities, health care providers, and other medical professionals. Though the consultation application 122 is shown by FIG. 1 in the server 120, in various embodiments, some or all of the consultation application's functionality is provided by a smartphone app or other software (not shown) executing on the patient computer device 110; in some embodiments, this software operates in communication and coordination with the virtual consultation application 122.

The patient computer device 110 may be connected to the network system 150 in order to access the consultation application 122. For example, a smartphone may connect to the consultation application 122 over the Internet through a software application, mobile app, or web portal. The consultation application 122 may require an authenticator, such as a username, password, or encryption key, before granting access to the patient 102. The consultation application 122 may be connected to the remote database 130 over the network system 150 to provide the consultation application 122 access to interactive care plans 140 and other data stored on the remote database 130.

The consultation application 122 receives patient symptom information 103 using the at least one patient computer device 110. For example, after a patient 102 logs into the consultation application 122, they may be prompted to describe their symptoms. The consultation application 122 may solicit these descriptions through visual, auditory, or tactile expressions. For example, the consultation application 122 may provide the patient 102 with an interactive diagram of the human body that allows the patient 102 to indicate the area where symptoms occur. As another example, the consultation application 122 may allow the patient 102 to enter symptom information 103 by voice or text. As another example, the consultation application 122 may allow the patient 102 to submit photographs of symptoms, for instance, using the camera on a smartphone. The photographs may be analyzed using machine learning to diagnose underlying medical conditions. The consultation application 122 may ask the patient 102 questions about their symptoms based on information provided by the patient 102, or based on the patient 102's medical history, seasonal medical issues, or epidemiological data. For instance, during flu season, the consultation application 122 may solicit symptom information 103 related to cold and flu symptoms. As another example, for a patient 102 with a history of chronic pain, the consultation application 122 may begin by soliciting symptom information 103 related to the chronic pain. As another example, if an illness is known to be spreading throughout a geographic region where the patient 102 is located, the consultation application may begin by soliciting symptom information 103 related to that illness. The consultation application 122 may ask follow-up questions about the symptom information 103. The follow-up questions may also include requests for photographs, measurements such as temperature, blood pressure, or heart rate, or tests such as blood panels.

The consultation application 122 may also solicit patient symptom information 103 from sources other than the patient 102. For example, if a caregiver or medical professional is using the consultation application 122 on behalf of the patient 102, the consultation application 122 may solicit information from the non-patient user. As another example, if the patient 102 has recently undergone any testing procedures at a medical facility, the consultation application 122 may solicit the test results from that facility. In still another example, the consultation application 122 may query the database or office of a remote medical provider for the medical history of a patient 102.

The consultation application 122 may receive patient symptom information 103 and store it within the remote database 130. The consultation application 122 may analyze the patient symptom information 103 to determine an appropriate interactive care plan 140 for treatment, matching the interactive care plan 140 to the patient 102's medical condition. Matching may be based on a number of factors. For example, the consultation application 122 may choose an interactive care plan 140 based on the presence and severity of symptoms reported by the patient. The consultation application 122 may match the reported symptoms with a list of symptoms treated by certain interactive care plans 140. In some cases, several interactive care plans 140, 141, and so on may match with the reported symptoms. As another example, the consultation application 122 may choose an interactive care plan 140 based on the patient 102's medical history and prognosis. For example, if the patient 102 has not been successful using certain treatments in the past, the consultation application may match a different interactive care plan 140 to the patient 102. The interactive care plan 140 may be matched based on other factors, such as the cost of treatment, the severity of treatment, the patient 102's likelihood of following the plan, and the like. The interactive care plan 140 may be matched based on any combination of the above factors. Furthermore, the interactive care plan 140 may be matched based on global or historical statistics determined by the consultation application 122. For instance, the consultation application 122 may keep a record of all patients who have been matched with a particular interactive care plan 140, whether they were successful, and other data relevant to the treatment process. The consultation application 122 may match interactive care plans 140 with higher success rates. In another example, the consultation application 122 may analyze the reasons contributing to the success of the interactive care plans 140, and may custom match plans to patients 102 based on those reasons.

After analyzing the patient symptom information 103 and determining an appropriate interactive care plan 140 for treatment, the consultation application 122 may recommend one or more interactive care plans 140 to the patient 102. The recommendation may include information about the likelihood of success, the cost of the plan, the difficulty of treatment, and the like. The consultation application 122 may present multiple interactive care plans 140, 141, and so on to the patient. The patient may be allowed to select one interactive care plan based on their treatment goals, cost sensitivity, or other prerogatives.

In one example, the consultation application 122 may recommend one or more interactive care plans 140 to a medical professional providing advice or treatment to the patient 102. The medical professional may review the plans and recommend one to the patient 102.

An interactive care plan 140 can be created for any health scenario, including prevention, wellness, management of chronic conditions, and post-discharge follow-up from a procedure or treatment.

The virtual consultation application 122 also recommends an interactive care plan 140 to the patient 102 that includes at least one of patient education, a virtual consultation with the health care provider, and an in-person consultation with the health care provider (not shown).

FIG. 2A is a flow chart for an exemplary interactive care plan based on symptom severity, according to the first embodiment of the present disclosure. In this example, the plan has three lines of treatment based on the severity of the patient's reported symptoms. The top line shows treatment based on symptoms that appear to be normal, i.e., within an acceptable range given appropriate medical standards. For instance if a person with diabetes were using the consultation application, a fasting blood glucose level below 100 mg/dl may be considered normal. The middle line shows treatment based on symptoms that appear to be moderate. For instance, if a diabetic's fasting blood glucose level was between 100-150 mg/dl, that may be considered moderate. The bottom line shows treatment based on symptoms that appear to be extreme. For instance if the diabetic's fasting blood glucose level was above 150 mg/dl, that may be considered extreme. The interactive care plan may contain any number and delineation of symptom severity treatments as are appropriate to provide proper treatment to the patient. Multiple symptoms may be considered together to determine the appropriate treatment path.

Following along the treatment line for “normal” symptoms in box 210, normal symptoms may not require pharmaceutical or other medical intervention. Therefore, the plan may call for patient education 212 in order to teach the patient about the medical condition, including what measurements are considered within a normal range. The patient education 212 may also include videos, books, podcasts, images, and other medical publications about the condition. This may be followed by monitoring 214 to ensure that the patient continues to exhibit normal metrics. Monitoring 214 may require the patient to check back every so often. For instance, a diabetic may be required to check their fasting blood glucose level periodically and report it to the consultation application. Monitoring 214 may require that the patient conduct a self-assessment for a period of time. The consultation application may send notifications and reminders to the patient to monitor their symptoms. These notifications and reminders may include e-mails, text messages, phone calls, push notifications, and other notifications commonly received on mobile devices. The notifications may be auditory, visual, tactile, or some combination thereof. If the symptoms remain normal, but the patient remains concerned, the patient may be given resources for further education 216 or may be referred for a virtual consult 218. Further education 216 may include tips for living with the medical condition, techniques for mitigating apparent symptoms, access to support groups or counselors, and the like. For example, a diabetic whose fasting blood glucose shows normal might benefit from an eating plan designed to regulate blood glucose levels or an appointment with a nutritionist. A virtual consult 218may include text or instant message conversations with a medical professional. The medical professional may discuss the patient's symptoms and potential treatments to discover if an alternative treatment plan may be more appropriate. A virtual consult 218 may also put the patient's mind at ease about their symptoms.

Following along the treatment line for “moderate” symptoms in box 220, moderate symptoms may initially require a virtual consult 222. The virtual consult 222 may be by text or instant message as above. The medical professional may advise pharmaceutical or other medical intervention for the symptoms. The medical professional may also advise that the patient refrain from taking any actions. The medical professional may recommend a period of monitoring 224 to observe whether the symptoms persist in the face of treatment or over time. If the symptoms return to normal levels, but the patient still has concerns about the condition, the treatment plan may then follow the “normal” treatment path of patient education 212, monitoring 214, further education 216, or virtual consult 218.

Following along the treatment line for “extreme” symptoms in box 230, extreme symptoms may initially require a phone triage 232. The phone triage 232 may be any type of real-time audio or video consultation with a medical professional. When the patient computer device is a mobile device, such as a smartphone or tablet, the patient may use the mobile device to conduct the consultation. Alternatively, the patient may be given a phone number or video conference link to connect to. During the phone triage 232, the medical professional may learn more about the patient and the severity of symptoms. The medical professional may ask follow-up questions or may require further measurements or tests. If the medical professional determines that the condition is serious enough, they may recommend that the patient have an in-person consultation 234 with a live medical professional. If the medical professional determines that the condition does not require an in-person consultation 234, they may recommend a virtual consultation 222 as described above. From there, the treatment path may then follow the “moderate” treatment path of virtual consult 222, monitoring 224, patient education 212, monitoring 214, further education 216, or virtual consult 218.

Relative to FIGS. 1 and 2, the interactive care plan 140 may include several components executed by the consultation application 122. One such component may be virtual care, including video consultations, voice consultations, instant messaging, text messaging, and e-mail. The virtual care component may allow a provider located remote from the patient 102 to provide medical treatment to the patient 102. The virtual care component may be included at one or more points in the interactive care plan 140. For example, as shown in FIG. 2A, a patient may communicate with a provider through phone triage 232, virtual consult 222, and virtual consult 218, depending on the severity and persistence of symptoms. The virtual care component may be customizable by the patient 102 or the provider. For example, some providers may wish to provide more instant messaging than voice consultations. As another example, some providers may provide more frequent virtual care for patients with more severe medical conditions or adverse medical histories.

Another component of the interactive care plan 140 may be the prescription of common health care treatment courses, including medical treatments, pharmaceutical treatments, exercises, testing, follow-up appointments, and the like. For example, after a patient 102 has received a virtual consult 222 or an in-person consult 234, the consulting provider may prescribe medication for the patient 102, and may include that in the interactive care plan 140. As another example, after consultation, the provider may refer the patient 102 to a specialist near the patient 102's geographic location, and may include that in the interactive care plan 140. Providers may select treatment courses from a list of courses included with one or more of the interactive care plans 140. Treatment courses may be authored by other providers and submitted to the database 130 for utilization by any providers using the consultation application 122.

Another component may be notifications, such as alerts and reminders. For example, an interactive care plan 140 may send daily reminders to a patient 102 to perform an exercise or take medication. Another interactive care plan 140 may send monthly notifications to a patient 102 and provider that a prescription should be refilled. Still another may send reminders to a patient 102's caregivers to prepare for a follow-up visit. In one example, notifications and other communications may be automatically suggested and sent by the consultation application 122. The consultation application 122 may determine how and when to send communications based on several factors, including the patient 102's history, the nature of the medical condition, and the like. The consultation application 122 may adjust the frequency and nature of communications in order to optimize patient engagement and plan adherence. For example, the consultation application 122 may increase the frequency of notifications to a patient 102 in order to increase the likelihood that the patient 102 will follow an interactive care plan 140. As another example, the consultation application 122 may send notifications to the patient 102 via mobile device, e-mail, and voice message to increase the likelihood that the patient 102 will follow the interactive care plan 140. Notifications may be sent to a patient 102's caregivers or other medical professionals as well. In one example, the consultation application 122 may automatically suggest the recipients, method, and frequency of notifications. For instance, the consultation application 122 may suggest that, where patients depend significantly on caregivers, all notifications should be sent to the caregivers. The interactive care plan 140 may also include reminders and notifications for upcoming or missed actions, and may automatically adjust their frequency and method of delivery. Notifications may be customized by the provider. For instance, some providers may want notifications sent more frequently. Some providers may assign more frequent notifications for more serious medical conditions, and fewer notifications for less serious medical conditions. Notification customization may also be based on a combination of multiple factors. Notifications may also be triggered by certain actions. For example, measurements or lab tests that return values in certain ranges may trigger notifications to be sent to a patient 102, caregiver, or provider. As another example, a notification may be sent to a provider when a patient 102 checks into a hospital or receives other emergency care.

The interactive care plan 140 may also include checklists or task lists. For example, an interactive care plan 140 may include a list of patient tasks, such as obtaining medication, resting, scheduling an appointment with a specialist, observing symptoms, and the like. The checklists or task lists may include any matter relevant to the patient's treatment. The checklists or task lists may be viewable by the patient, the patient's caregivers, other medical professionals, and the provider. Checklists or task lists may list different tasks, depending on the recipient. For example, a task list directed to a patient 102 may include the task of scheduling an appointment, while a task list directed to a caregiver may include the task of reminding the patient 102 to schedule an appointment. The consultation application 122 may automatically determine which tasks to send when multiple recipients are involved.

The treatments, notifications, checklists, and other aspects of the interactive care plan 140 may be customizable based on the provider 302 and the patient. In one example, the consultation application 122 may automatically choose or suggest the components, or portions of the components of the interactive care plan 140. The consultation application 122 may determine the components using results from data analytics. In one example, an analytics module and accompanying services may allow the querying of all events data, both historic and real-time. The analytics module may automatically reason around patterns and rates related to patient behavior. The analytics module may calculate the appropriate combination of treatments, notifications, checklists, and other components to optimize patient engagement and adherence to an interactive care plan. In another example, the consultation application 122 may employ situational awareness analytics, allowing it to aggregate, query, and analyze data, and learn from data over time.

In one example, interactive care plans 140 may be consistent with current clinical guidelines. In other words, interactive care plans 140 may be based primarily on consensus and standards within the medical community about patient care and treatment for a particular medical condition. Interactive care plans 140 may improve care by enabling healthcare providers to guide their patients through healthcare decisions tailored to individual patients 102. Interactive care plans 140 also may encourage successful treatment by incorporating room for customer preference. Compliance with interactive care plans 140, especially for common care episodes, may be mutually beneficial to patients 102, caregivers, families, payors, and providers within contained health ecosystems. Increased compliance may contribute to improved clinical outcomes for the patient 102, which may lead to improved health and lower costs of care.

In another example, interactive care plans 140 may operate according to defined intervention parameters. For instance, one intervention parameter may include event triggers. Events may be triggered by external systems, such as hospitalization, lab test results, and the like. Events may also be triggered by internal systems, such as reported symptoms, missed appointments, new health metrics, and the like. Triggered events may cause a certain interactive care plan 140 to be recommended, or they may cause certain paths of an interactive care plan 140 to be recommended. Another intervention parameter may be resulting actions. An interactive care plan 140 may prompt one or more actions based on the duration, frequency, expiration time, or deadline for the action. This may cause the interactive care plan 140 to alert a user about the action, request data, such as a test or measurement, schedule a consultation or visit, or provide information to a user. Another intervention parameter may be scoring criteria, which determines how to measure progress along an interactive care plan 140. This parameter may define metrics and ranges for components of the interactive care plan 140, and may trigger portions of the interactive care plan 140 according to the calculated score.

Relative to FIGS. 1 and 2, several types of users may access the consultation application 122.

As one example, health care providers may access the consultation application 122 to perform medical services, check in on patients 102, recommend interactive care plans 140, or modify interactive care plan 140 rules. For instance, when performing virtual consultations 218, 222, providers may be signed into the consultation application 122. They may receive a notification that a patient 102 has requested the consultation and may elect to perform the consultation. As another example, providers may use the consultation application 122 to check a patient's progress against tasks assigned by the interactive care plan 140. The provider may be able to see which tasks the patient 102 has accomplished and which remain outstanding. As another example, the provider may use the consultation application 122 to search a database 130 of interactive care plans 140, 141, and so on. The database 130 may contain a number of interactive care plans 140, 141, and so on submitted by other providers. The consultation application 122 may perform analysis

As another example, a provider may wish to modify the interactive care plan 140 for a patient based on the patient 102's compliance, success of the treatment methodology, or other factors. The provider may access the interactive care plan 140 through the consultation application 122 to make adjustments. In still another example, the provider may use the consultation application 122 to schedule referral consultations, surgeries, or other medical procedures as part of an interactive care plan 140 for a patient 102.

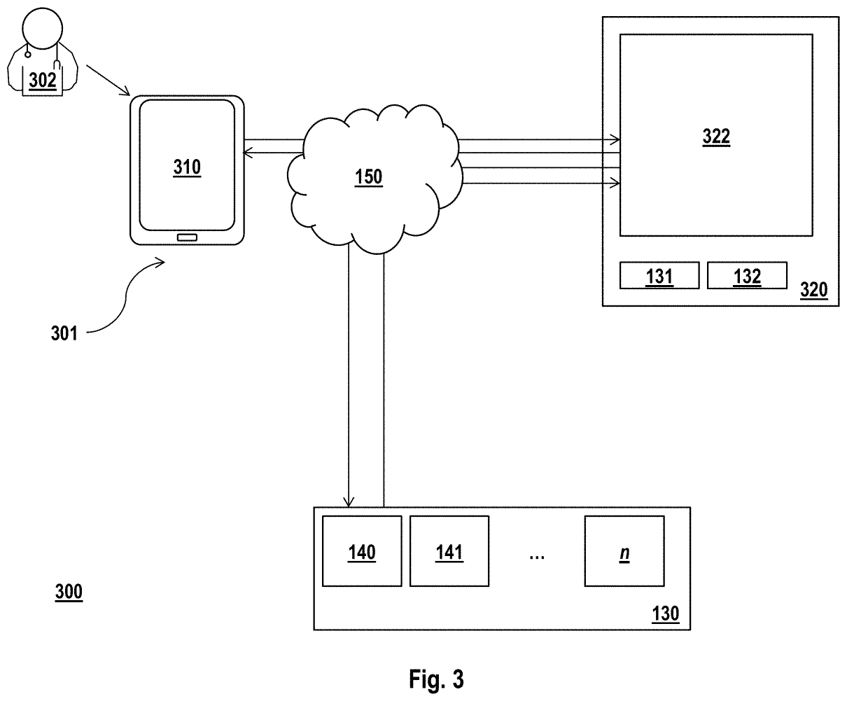

Providers may also author interactive care plans 140 using a virtual authoring application, which is discussed in greater detail in FIG. 3.

As another example, health care providers, case managers, and administrators (hereinafter “administrators”) may access the consultation application 122 to perform monitoring, optimization, and analytics. For instance, the consultation application 122 may collect data, such as patient data, treatment data, and compliance data. Patient data may include patient information such as age, weight, geographic location, existing medical conditions, and the like. Patient data may be organized according to various patient groups, or cohorts. For example, certain cohorts may divide patients 102 based on age ranges, geographic locations, demographics, comorbidities, adherence levels, and other medical factors. Some cohorts may divide patients 102 based on multiple categories of patient data. Cohorts may be created automatically by the consultation application 122 or may be customizable by administrators. Treatment data may include information such as the nature of treatment, when treatment was prescribed, the results of treatment, and the like. Compliance data may include information such as how patients 102 complied with treatment, how often compliance resulted in successful treatment, and the like. The consultation application 122 may collect other data relevant to patient care. The data may be stored in the database 130. The consultation application 122 may process one or more sets of data for use by administrators. For instance, administrators wishing to monitor the success rates of interactive care plans 140 may use a tool within the consultation application 122 to track treatment data. Administrators may, for example, track overall success rates within the network, or they may track success rates relating to specific cohorts. As another example, administrators may use the consultation application 122 to optimize interactive care plans 140 for various cohorts. For instance, the consultation application 122 may compare the success of treatment or compliance rates for multiple interactive care plans 140, 141 to determine which are more effective. Administrators may use the cohort data to determine which factors lead to increased effectiveness among different groups, and may assign ratings to interactive care plans 140 based on their successfulness. The consultation application 122 may analyze the data in detail, providing suggestions for effective interactive care plans 140 specifically tailored to individuals. Administrators may also use the consultation application 122 to create AB-type tests that compare interactive care plans 140, 141 directly across a number of variants.

As another example, patients 102 may access the consultation application 122 to receive medical care, view checklists and task lists, and to track progress. For instance, patients 102 seeking medical care may use the consultation application 122 to receive triage assessments and progress through an interactive care plan 140 as described in FIG. 2A. The consultation application 122 may provide a library of medical information related to a patient 102's medical condition that may be accessed to educate the patient 102. In one example, a patient 102 may even access provider opinions from specific providers or opinions directed specifically to their issues. A patient 102 may use the consultation application 122 as a platform for receiving virtual care via phone, text, video, or other interface. Additionally, the consultation application 122 may be used as a platform for scheduling appointments and procedures. Patients 102 viewing task lists may access the consultation application 122. Task lists may have interactive components, such as marking tasks “completed”, following interactive links for scheduling, learning, or prescription ordering, and the like. Patients 102 may also access the consultation application 122 to track their progress. For example, the consultation application 122 may tell a patient 102 how well they are adhering to an interactive care plan 140, along with how much progress they have made along the plan. As another example, the consultation application 122 may display a patient 102's health metrics over time for review by the patient 102. As another example, a patient 102 may use the consultation application 122 to track their progress toward health goals. For instance, a patient 102 who has pledged to lose weight may use the consultation application 122 to view their historical weight as recorded by a connected scale. A patient 102 who has pledged to reduce his cholesterol may view his historical cholesterol measurements. Patients 102 may be alerted by the consultation application 122 when they have progressed along these goals. As another example, a patient 102 may access the consultation application 122 to review and receive notifications according to an interactive care plan 140, patient preference, or provider preference.

As still another example, caregivers may access the consultation application 122 to receive notifications or provide support to patients 102. Caregivers may include medical staff, assisted living staff, family, friends, or others who assist the patient 102 with their medical care. Caregivers receiving notifications may access the consultation application 122 using a patient computer device 110. Caregivers may view notifications through an inbox or other message platform. In one example, caregivers may be able to interact with notifications, for instance by communicating with a provider regarding the notification, marking a task as “completed”, or scheduling an appointment. Caregivers may access the consultation application 122 to provide other patient support. In one example, caregivers may be directed to a support dashboard where they can view patient performance, adherence to interactive care plans 140, progress along health goals, and the like. Caregivers may be able to communicate with patients 102 using the consultation application 122. Communications may be used to provide motivational support, remind patients 102 of upcoming tasks or appointments, discuss test results, and the like. This may improve the support that caregivers located remote from patients 102 provide.

The following examples are provided to illustrate exemplary situations where interactive care plans in accordance with the subject disclosure are used in conjunction with specific medical circumstances.

### EXAMPLE 1

FIG. 2B is a flow chart for an exemplary interactive care plan for a pregnant patient who is pre-diabetic. The boxes in the upper left of the chart show the regular state care plan for the patient, including providing the patient with daily and weekly information, scheduling an in-person consult every 6 months, scheduling a virtual consult every 3 months, using a glucometer several times per week, and taking Metformin to control blood glucose. On Day 1, the patient becomes pregnant, and seeks a consult using the consultation application.

Based on the severity of her blood glucose levels, she may be placed onto one of three treatment paths under the interactive care plan. If presenting with “normal” blood glucose levels, she may be directed to test to confirm the pregnancy. If the pregnancy test is positive, she will be directed to have a virtual consult. The virtual consult may lead the patient along several treatment paths. If her weight is higher than expected, the patient may be directed to have a virtual consult with a nutritionist and given information about gestational diabetes relevant to her pregnancy. In another treatment path, the consulting provider will require lab tests and measurements. If the patient is shaky, achy, or excessively hungry, she will be directed to have a virtual consult for blood glucose. If her blood glucose measurements are high, she will be directed to have a virtual consult with a physician. The physician may prescribe an insulin product in place of the patient's current Metformin prescription. If the patient's blood glucose measures high for 3 consecutive days, the patient will be directed to have a phone triage, which will lead to a virtual consult with a physician. The physician may recommend increasing the dosage of the insulin product. If the patient's blood glucose measurements are very high for 3 consecutive days, she will be directed to use a glucometer and have a virtual consult with a physician. The patient may also undergo a phone triage and a virtual consult with a physician, who may recommend an insulin product. As a result of the earlier lab tests, the patient may be placed on an interactive treatment plan that has been modified from her regular state care plan. This plan may include a modified Metformin prescription, the use of a glucometer several times a week, weight measurements weekly, virtual consults with a nurse every 3 weeks, virtual consult with an obstetrician at 3 months of pregnancy, and daily and weekly information sent to the patient. This may continue for the duration of the patient's pregnancy.

If presenting with “moderately concerning” blood glucose levels, the patient may be directed to have a virtual consult with a physician and given information on gestational diabetes relevant to her pregnancy. If presenting with “extremely concerning” blood glucose levels, the patient may be directed to immediate phone triage. If a virtual consult is sufficient, the patient will have a virtual consult with a physician. If immediate in-person care is needed, the patient will be told to go to an urgent care facility.

Once the patient is longer pregnant, the interactive care plan may return to the regular state care plan, or may be modified to a post-gestation plan.

### EXAMPLE 2

FIG. 2C is a flow chart for an exemplary interactive care plan for a patient with mild Chronic Obstructive Pulmonary Disease. The boxes in the top left show her regular state interactive care plan, which includes the use of Symbicort medication twice daily, weekly spirometer and pulse oximeter use, weekly symptom survey, a virtual consult with a physician every 3 months, an in-person consult with a physician every 6 months, and daily and weekly information. One Day 1, the patient presents with a chest cough, and seeks a consult using the consultation application.

Based on the severity of her symptoms, the patient will be directed to one of three treatment paths. If presenting with “normal” symptoms, the patient will be directed to a digital library to learn more about her condition. If she is still concerned, the patient may be directed to have a text virtual consult with a physician and given more opportunities for education over the next several weeks. If presenting with “moderately concerning” symptoms, the patient will be directed to have a virtual consult with a physician. The physician may prescribe short-term medication, may require that patient measurements be taken more frequently, and may provide daily and weekly education for the patient. If the patient's symptoms do not improve over several days, she will have another virtual consult with a physician. The physician may order lab tests, nebulizers, and additional medication for the patient. After several days, the patient may have further virtual consults until her symptom measurements return to their regular state. If presenting with “extremely concerning” symptoms, the patient may be directed to have a phone triage appointment to understand the symptoms better. If virtual consult is sufficient, the patient will have a virtual consult with a physician. If immediate in-person care is needed, the patient will be told to go to an urgent care facility.

Once the patient's chest cough subsides, she may be returned to her regular state interactive care plan.

FIG. 3 is a box diagram 300 showing a computer-implemented system for delivering an interactive care plan to a patient from a health care provider located remote from the patient, according to a second exemplary embodiment of the present disclosure. The system 301 includes a provider computer device 310 and a remote database 150. A virtual authoring application 322 hosted at least partially on a server 320 having a processor 131 and computer-readable memory 132 is electronically accessible over at least one network system 150 by the provider computer device 310 and the remote database 130. The virtual authoring application 322 receives an interactive care plan 140 from the health care provider 302 using the provider computer device 310. The interactive care plan 140 is stored on the remote database 130. The interactive care plan 140 is accessible to a patient from the remote database 130.

The provider computer device 310 may be any electronic or computer device that provides an interface between the provider 302 and the virtual authoring application 322. For example, the provider computer device 310 may be a personal computer or a mobile device, such as a tablet or a smartphone. The provider 302 may use this device to author, edit, annotate, and otherwise manipulate an interactive care plan through the virtual authoring application 322. The provider computer device 310 may include components generally found in a computer or mobile device, such as a processor, computer-readable memory, a screen or monitor, an input interface, and network connection hardware. The provider computer device 310 may allow a provider 302 to interface with the virtual authoring application 322 using a mouse and keyboard, touch interface, audio interface, visual interface, or any combination thereof.

The virtual authoring application 322 (hereinafter “authoring application” 322) may be a software application hosted at least partially on a server. The authoring application 322 may provide a user interface, including a graphical user interface, for the provider 302 when authoring an interactive care plan 140. The authoring application 322 may be accessible over at least one network system 150. The authoring application 322 may utilize encryption protocols over the network system 150, such as LTS or SSL, to establish a secure connection and limit access to sensitive medical information.

The provider computer device 310 may be connected to the network system 150 in order to access the authoring application 322. For example, a tablet or personal computer may connect the authoring application 322 over the Internet through a software application, mobile app, or web portal. The authoring application 322 may require an authenticator, such as a username, password, or encryption key, before granting access to the provider 302. Though the authoring application 322 is shown by FIG. 3 in the server 320, in various embodiments, some or all of the authoring application's functionality is provided by a smartphone app or other software (not shown) executing on the provider computer device 310; in some embodiments, this software operates in communication and coordination with the authoring application 322.

The provider 302 may use the provider computer device 310 and the authoring application 322 to create and edit an interactive care plan 140. For example, the authoring application 322 may provide a graphical interface where a provider 302 may create and organize components of an interactive care plan 140. The components of the interactive care plan 140 may be based on medical standards, new treatment theories, or best practices. The provider 302 may include several points of customization in the interactive care plan 140 for optimal adherence by one or more cohorts. For example, using analytic data developed by a consultation application, the provider 302 may determine that certain cohorts are treated more successfully by interactive care plans 140 that include more frequent notifications. The provider 302 may include options for additional notifications in the interactive care plan 140. As another example, a provider 302 may customize an interactive care plan 140 with multiple levels of treatment options based on the severity of symptoms, cost of treatment, or other factors. The provider 302 may use the authoring application 322 to edit a previously-created interactive care plan 140. For instance, using data stored on the database 130, the provider 302 may determine that a component of an interactive care plan 140 is ineffective. The provider 302 may use the authoring application 322 to change an aspect of that component in order to improve effectiveness.

Providers 302 may also use the authoring application 322 to submit interactive care plans 140 to the database 130 for use by patients and other providers. The authoring application 322 may include a platform for identifying the submitting provider 302, tracking the usage of an interactive care plan 140, or providing financial compensation to a provider 302 for submitting an interactive care plan 140. Providers 302 may also use the authoring application 322 to track and monitor their submitted interactive care plans 140.

In one example, the authoring application 322 may be a tool provided within the consultation application. For instance, the consultation application may include a provider portal that limits access to health care providers 302. From that portal, providers 302 may access the authoring application 322. In another example, the authoring application 302 is separate from the consultation application.

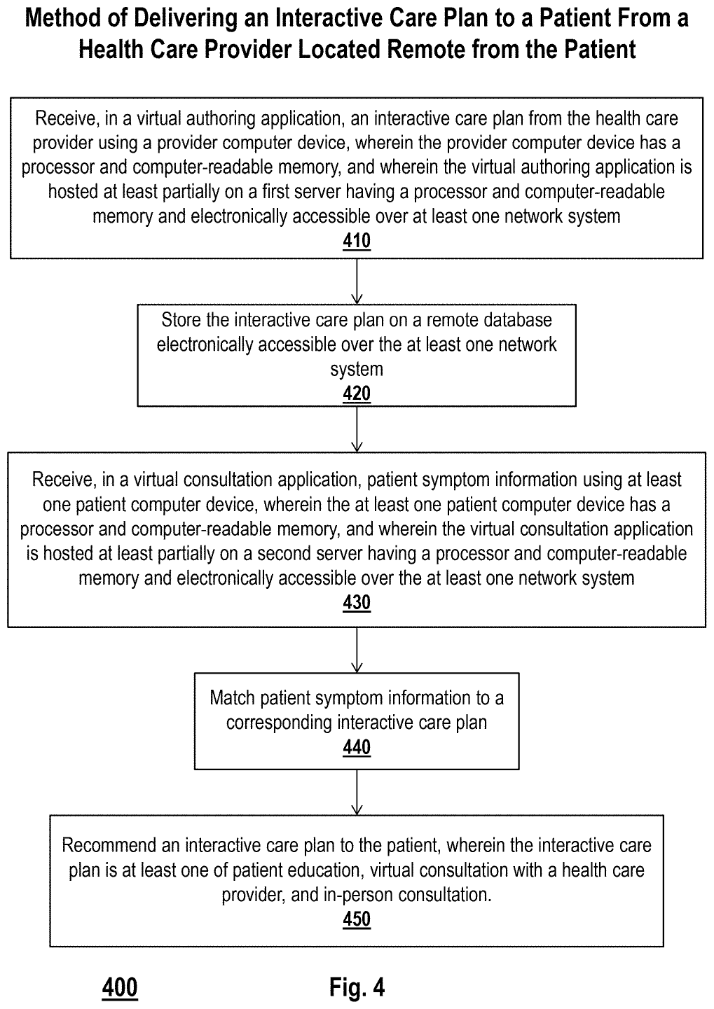

FIG. 4 is a flowchart 400 illustrating a method of delivering an interactive care plan to a patient from a health care provider located remote from the patient. It should be noted that any process descriptions or blocks in flow charts should be understood as representing modules, segments, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As shown in box 410, an interactive care plan is received from the health care provider using the provider computer device, wherein the provider computer device has a processor and computer-readable memory. The interactive care plan is received in a virtual authoring application hosted at least partially on a first server having a processor and computer-readable memory and electronically accessible over at least one network system.

As shown in box 420, the interactive care plan is stored on a remote database electronically accessible over the at least one network system.

As shown in box 430, patient symptom information is received using at least one patient computer device, wherein the at least one patient computer device has a processor and computer-readable memory. The patient symptom information is received in a virtual consultation application hosted at least partially on a second server having a processor and computer-readable memory and electronically accessible over the at least one network system. In one example, the first server in box 410 and the second server in box 430 may be separate servers. In another example, the first server and the second server may be the same server.

As shown in box 440, patient symptom information is matched to a corresponding interactive care plan.

As shown in box 450, an interactive care plan is recommended to the patient, wherein the interactive care plan is at least one of patient education, virtual consultation with a health care provider, and in-person consultation.

The method may further include any other features, components, or functions disclosed relative to any other figure of this disclosure.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A system for providing an interactive care plan to a patient, comprising a patient computer device comprising a processor, computer-readable memory, and one or more hardware components configured to receive symptom information for the patient; and
a server distinct from the patient computer device, the server having a processor and computer-open readable memory, the server and the patient computer device being accessible to one another via at least one network system, the server at least partially hosting a virtual consultation application, the virtual consultation application:
receiving from the patient computer device symptom information for the patient received by the patient computer device;
accessing a plurality of interactive care plans each specifying one or more measures to be taken on a patient's behalf;
using the received symptom information for the patient to select one of the plurality of accessed interactive care plans; and
recommending the selected interactive care plan to the patient via the patient computer device.

2. The system of claim 1 wherein the interactive care plans are stored in and accessed from the server.

3. The system of claim 1, further comprising a computing node hosting a database, the computing node being distinct from the server and the patient computer device, the database storing and providing access to the interactive care plans.

4. The system of claim 1 wherein one of the hardware components for receiving symptom information for the patient is a sensor configured to sense symptom information.

5. The system of claim 1 wherein one of the hardware components for receiving symptom information for the patient is a user input mechanism configured to receive symptom information specified by the patient by interacting with the user input mechanism.

6. The system of claim 1 wherein the selected interactive care plan specifies a patient education measure, a virtual healthcare provider consultation measure, or an in-person healthcare provider consultation measure.

7. The system of claim 6 wherein the virtual consultation application further analyzes the received symptom information for the patient to determine a severity level, and wherein the selection of the selected interactive care plan among client education measure, virtual healthcare provider consultation measure, and in-person healthcare provider consultation measure is based on the determined severity level.

8. The system of claim 1 wherein the accessed plurality of interactive care plans each have patient symptom information criteria, and wherein the virtual consultation application selects one of the plurality of accessed interactive care plans by matching the received symptom information for the patient against the patient symptom information criteria of the interactive care plans.

9. The system of claim 8 wherein the patient symptom information criteria of the interactive care plans include both symptom presence and symptom severity.

10. The system of claim 1 wherein the virtual consultation application selects one of the plurality of accessed interactive care plans based on patient medical history information.

11. The system of claim 1 wherein the virtual consultation application selects one of the plurality of accessed interactive care plans based on a success rate determined for prior recommendation of each of at least a portion of the plurality of interactive care plans to other patients.

12. A system for providing an interactive care plan to a patient, comprising
a healthcare provider computer device comprising a processor, computer-readable memory, and one or more input mechanisms configured to receive interactive care plan information specified by a healthcare provider; and
a server distinct from the healthcare provider computer device, the server having a processor and computer-open readable memory, the server and the patient computer device being accessible to one another via at least one network system, the server at least partially hosting a virtual authoring application, the virtual authoring application:
receiving from the healthcare provider computer device interactive care plan information specified by a healthcare provider;
using the received interactive care plan information to create an interactive health plan on the healthcare provider's behalf that specifies one or more measures to be taken on a patient's behalf;
storing the created interactive health plan in an interactive health plan repository in which it is accessible to a patient.

13. The system of claim 12 wherein the interactive health plan repository is on the server.

14. The system of claim 12, further comprising a computing node hosting a database, the computing node being distinct from the server and the patient computer device, the database containing the interactive health plan repository.

15. The system of claim 12 wherein the interactive health plan created by the virtual authoring application contains a patient symptom criterion specified by the healthcare provider.

16. The system of claim 15 wherein the patient symptom criterion contained by the created interactive care plan specify both symptom presence and symptom severity.

17. The system of claim 12 wherein the interactive health plan created by the virtual authoring application contains a patient medical history criterion specified by the healthcare provider.

18. A method in a computing system for providing an interactive care plan to a patient, the method comprising:
receiving via a healthcare provider computer device interactive care plan information specified by a healthcare provider;
using the received interactive care plan information to create an interactive health plan on the healthcare provider's behalf that specifies one or more measures to be taken on a patient's behalf;
adding the creative interactive health plan to an interactive health plan repository in which it is accessible to patients;
receiving via a patient computer device patient condition information for a patient;
accessing a plurality of interactive care plans in the interactive health plan repository;
using the received condition information for the patient to select one of the plurality of accessed interactive care plans; and
recommending the selected interactive care plan to the patient via the patient computer device.

19. The method of claim 18 wherein the selected interactive care plan specifies a patient education measure, a virtual healthcare provider consultation measure, or an in-person healthcare provider consultation measure.

20. The method of claim 19, further comprising analyzing the received symptom information for the patient to determine a severity level, and wherein the selection of the selected interactive care plan among client education measure, virtual healthcare provider consultation measure, and in-person healthcare provider consultation measure is based on the determined severity level.

21. The method of claim 18 wherein the accessed plurality of interactive care plans each have patient condition information criteria, and wherein one of the plurality of accessed interactive care plans his selected by matching the received condition information for the patient against the patient condition information criteria of the interactive care plans.

22. The method of claim 21 wherein the patient condition information criteria of the interactive care plans include both condition presence and condition severity.

23. The method of claim 18 wherein one of the plurality of accessed interactive care plans is selected based on patient medical history information.

24. The method of claim 18 wherein one of the plurality of accessed interactive care plans is selected based on a success rate determined for prior recommendation of each of at least a portion of the plurality of interactive care plans to other patients.
